(19) United States
(12) Patent Application Publication
(10) Pub. No.: US 2018/0264281 A1
(43) Pub. Date: Sep. 20, 2018

(54) MICROWAVE OUTPUT METHOD FOR THERMOTHERAPY AND APPARATUS FOR PERFORMING THE SAME

(71) Applicant: Electronics and Telecommunications Research Institute, Daejeon (KR)

(72) Inventors: Jang Yeol KIM, Daejeon (KR); Seong-Ho SON, Daejeon (KR); Soon Ik JEON, Daejeon (KR); Bo Ra KIM, Daejeon (KR); Kwang Jae LEE, Daejeon (KR)

(73) Assignee: Electronics and Telecommunications Research Institute, Daejeon (KR)

(21) Appl. No.: 15/684,446

(22) Filed: Aug. 23, 2017

(30) Foreign Application Priority Data
Mar. 20, 2017 (KR) 10-2017-0034803

(51) Int. Cl. A61N 5/02

(52) U.S. Cl. CPC A61N 5/025

(57) ABSTRACT

Disclosed is a microwave output method and apparatus for thermotherapy. The microwave output method may include performing a forward analysis using a mapping model corresponding to a target point of an object, performing a reverse analysis based on forward analysis data for the forward analysis, and outputting a signal to the target point from each of a plurality of antennas based on reverse analysis data for the reverse analysis.

### CROSS-REFERENCE TO RELATED APPLICATION(S)

This application claims the priority benefit of Korean Patent Application No. 10-2017-0034803 filed on Mar. 20, 2017, in the Korean Intellectual Property Office, the disclosure of which is incorporated herein by reference for all purposes.

### BACKGROUND

### 1. Field

One or more example embodiments relate to a microwave output method and apparatus for thermotherapy based on a noninvasive method using a microwave.

### 2. Description of Related Art

In general, a method of curing a tumor includes a surgery, a radiotherapy, a pharmacotherapy, and a hormone therapy. However, such methods may be unable to remove or necrotize tumor cells completely and cause many side effects. Thus, methods of combining a surgery, a radiotherapy, a pharmacotherapy, and a hormone therapy are preferable. Such methods of combination may remove or necrotize more number of tumor cells without side effects.

Research is being done on thermotherapy for curing a tumor by increasing a temperature of a tumor portion as a method of necrotizing tumor cells by creating a synergy effect with a radiation therapy and a chemotherapy by many research groups.

Main research results based on a treatment algorithm of increasing a temperature of a target point by allowing a microwave to be focused on the inside a human body have been announced recently. Particularly, results of researches into focusing a microwave at a target point inside a human body using a time reversal (TR) algorithm of sound and ultrasonic wave technologies have been announced.

Apparatuses and methods of curing a disease, for example, a tumor, inside a human body and necrotizing tumor cells in response to a temperature being greater than or equal to a reference value by applying a TR algorithm and a microwave signal without a surgery or an incision using a noninvasive method other than an invasive method are requested.

### SUMMARY

An aspect provides a technology for obtaining a signal from a target point through a forward analysis.

Another aspect also provides a technology for outputting a signal to a target point through a reverse analysis.

Still another aspect also provides a technology for monitoring a target point.

According to an aspect, there is provided a microwave output method including a microwave output method including generating a mapping model corresponding to an object by mapping at least one of a permittivity or a conductivity to an image of the object, and performing a forward analysis on a target point based on a time reversal (TR) algorithm using the mapping model for allowing a signal to be focused on the target point of the object through a plurality of antennas.

The performing of the forward analysis may include setting a condition for a transmission antenna and a reception antenna based on the mapping model, and measuring a signal received by the reception antenna based on the condition.

The setting may include disposing the transmission antenna at the target point.

The setting may include disposing the reception antenna outside the target point, and the reception antenna is provided as a plurality of antennas.

The condition may include at least one of a position of the transmission antenna, a position of the reception antenna, a frequency of the signal, a type of the signal, or an analysis time.

The performing of the forward analysis may include representing an area corresponding to the target point to the mapping model based on the permittivity or the conductivity.

According to another aspect, there is provided a microwave output method including receiving signals received from a target point of an object, and outputting a signal to the target point from each of a plurality of antennas by performing a reverse analysis on the received signals based on a time reversal (TR) algorithm.

The outputting may include determining at least one of an amplitude or a phase of the signal based on at least one of an attenuation or a loss.

The determining comprises determining the amplitude of the signal to be less than or equal to a threshold value set to prevent surrounding tissue damage of the target point and determining the phase of the signal as opposed to a phase of the receiving signals received from the target point of the object.

The microwave output method may further include monitoring a temperature of the object.

The microwave output method may further include decreasing the temperature by readjusting the threshold value in response to a temperature of an area excluding the target point in the object being greater than or equal to a first reference value.

The microwave output method may further include re-performing the reverse analysis based on the TR algorithm by changing a condition in response to a temperature of the target point in the object being less than or equal to a second reference value.

According to still another aspect, there is provided a microwave output method including performing a forward analysis based on a time reversal (TR) algorithm using a mapping model corresponding to a target point of an object, performing a reverse analysis based on the TR algorithm based on forward analysis data for the forward analysis, and outputting a signal to the target point from each of a plurality of antennas based on reverse analysis data for the reverse analysis.

The performing of the forward analysis may include setting a condition for a transmission antenna and a reception antenna based on the mapping model, and measuring a signal received by the reception antenna based on the condition.

The setting may include disposing the transmission antenna at the target point.

The setting may include disposing the reception antenna outside the target point, and the reception antenna is provided as a plurality of antennas.

The condition may include at least one of a position of the transmission antenna, a position of the reception antenna, a frequency of the signal, a type of the signal, or an analysis time.

The performing of the forward analysis may include generating the mapping model by mapping a permittivity or a conductivity to an image of the object, and representing an area corresponding to the target point to the mapping model based on the permittivity or the conductivity.

The performing of the reverse analysis may include determining at least one of an amplitude or a phase of the signal based on at least one of an attenuation or a loss.

The determining comprises determining the amplitude of the signal to be less than or equal to a threshold value set to prevent surrounding tissue damage of the target point and determining the phase of the signal as opposed to a phase of the receiving signals received from the target point of the object.

The microwave output method may further include monitoring a temperature of the object.

The microwave output method may further include decreasing the temperature by readjusting the threshold value in response to a temperature of an area excluding the target point in the object being greater than or equal to a first reference value, and re-performing the reverse analysis based on the TR algorithm by changing a condition in response to a temperature of the target point in the object being less than or equal to a second reference value.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a block diagram illustrating a microwave output system according to an example embodiment;

FIG. 2 is a block diagram illustrating a signal obtaining apparatus of FIG. 1 according to an example embodiment;

FIG. 3 illustrates an antenna array of FIG. 1 according to an example embodiment;

FIG. 4 is a block diagram illustrating a focusing apparatus of FIG. 1 according to an example embodiment;

FIG. 5 is a flowchart illustrating a microwave output method according to an example embodiment;

FIG. 6 is a flowchart illustrating a mapping model obtaining method according to an example embodiment;

FIG. 7A illustrates a permittivity mapping model according to an example embodiment;

FIG. 7B illustrates a conductivity mapping model according to an example embodiment;

FIG. 8 is a flowchart illustrating a forward analysis according to an example embodiment;

FIG. 9 is a flowchart illustrating a method using a microwave output result according to an example embodiment; and

FIG. 10 is a flowchart illustrating a method of comparing a measurement result to a simulation result according to an example embodiment.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions may not be described in detail because they would obscure the description with unnecessary detail.

The terminology used herein is for the purpose of describing the example embodiments only and is not intended to be limiting of the disclosure. As used herein, the singular forms “a,” “an,” and “the” are intended to include the plural forms as well, unless the context clearly indicates otherwise. It will be further understood that the terms “include/comprise” and/or “have,” when used in this disclosure, specify the presence of stated features, integers, steps, operations, elements, components, or combinations thereof, but do not preclude the presence or addition of one or more other features, integers, steps, operations, elements, components, and/or groups thereof. In addition, the terms such as “unit,” “-er (-or),” and “module” described in the specification refer to an element for performing at least one function or operation, and may be implemented in hardware, software, or the combination of hardware and software.

Terms such as first, second, A, B, (a), (b), and the like may be used herein to describe components. Each of these terminologies is not used to define an essence, order or sequence of a corresponding component but used to distinguish the corresponding component from other component(s). For example, a first component may be referred to a second component, and similarly the second component may also be referred to as the first component.

It should be noted that if it is described in the specification that one component is “connected,” “coupled,” or “joined” to another component, a third component may be “connected,” “coupled,” and “joined” between the first and second components, although the first component may be directly connected, coupled or joined to the second component.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

A module used herein may indicate hardware that performs an operation and a function according to each term described herein, a computer program code to perform a predetermined function and an operation, or an electronic recording medium, for example, a processor or a microprocessor, including a computer program code to perform a predetermined function and an operation.

That is, a module may indicate hardware for performing a technical idea of the present invention and/or a functional and/or structural combination for driving the hardware.

Hereinafter, example embodiments are described in detail with reference to the accompanying drawings. Like reference numerals in the drawings denote like elements, and a known function or configuration will be omitted herein.

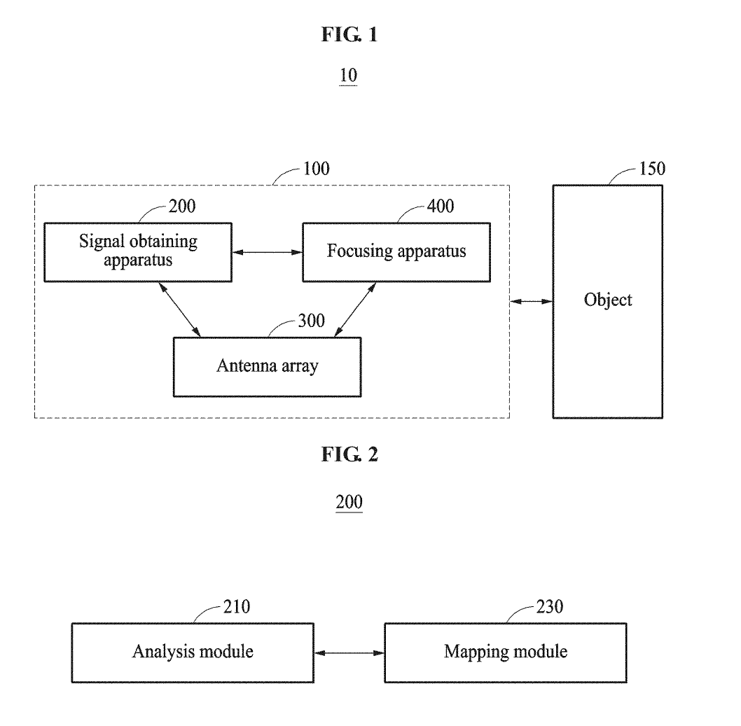

FIG. 1 is a block diagram illustrating a microwave output system according to an example embodiment.

In FIG. 1, a microwave output system 10 includes a microwave output apparatus 100 and an object 150.

The microwave output apparatus 100 outputs a microwave signal to the object 150. For example, the object 150 may be a body portion, for example, an arm and a leg of a human, a portion of an animal, or a portion of a robot. However, the object 150 is not limited thereto.

The microwave output apparatus 100 may be used to cure a target point by allowing a radio wave, for example, a microwave, to be focused on the target point of the object 150. The target point includes a tumor portion of a predetermined human body portion. That is, the microwave output apparatus 100 may be an apparatus for thermotherapy based on a noninvasive method using a microwave.

The microwave output apparatus 100 includes a signal obtaining apparatus 200, an antenna array 300, and a focusing apparatus 400.

The signal obtaining apparatus 200 performs a forward analysis using a mapping model corresponding to the target point of the object 150. The mapping model may be a phantom model corresponding to the target point, for example, a predetermined body portion, of the object 150. For example, the signal obtaining apparatus 200 performs a forward analysis using a plurality of antennas (or a plurality of transceivers) included in the antenna array 300.

The signal obtaining apparatus 200 transmits forward analysis data to the focusing apparatus 400.

The focusing apparatus 400 performs a reverse analysis based on the forward analysis data. Also, the focusing apparatus 300 may output a signal, for example, a microwave, to the target point through the antennas (or transceivers) included in the antenna array 300 based on the reverse analysis data for the reverse analysis.

Although FIG. 1 illustrates that the signal obtaining apparatus 200 and/or the antenna array 300 are provided outside the focusing apparatus 400, the signal obtaining apparatus 200 and/or the antenna array 300 are not limited thereto. The signal obtaining apparatus 200 and/or the antenna array 300 may be provided inside the focusing apparatus 400. The antenna array 300 may be provided inside the signal obtaining apparatus 200.

As described above, the microwave output apparatus 100 may be used to cure a disease, for example, a tumor, by allowing a beam (or signal) to be focused on a selectively desired target point using a radio wave, for example, a microwave, to be focused on a deep core of internal human tissues using a noninvasive method without a surgery or an incision, unlike a ultrasonic wave and an acoustic signal.

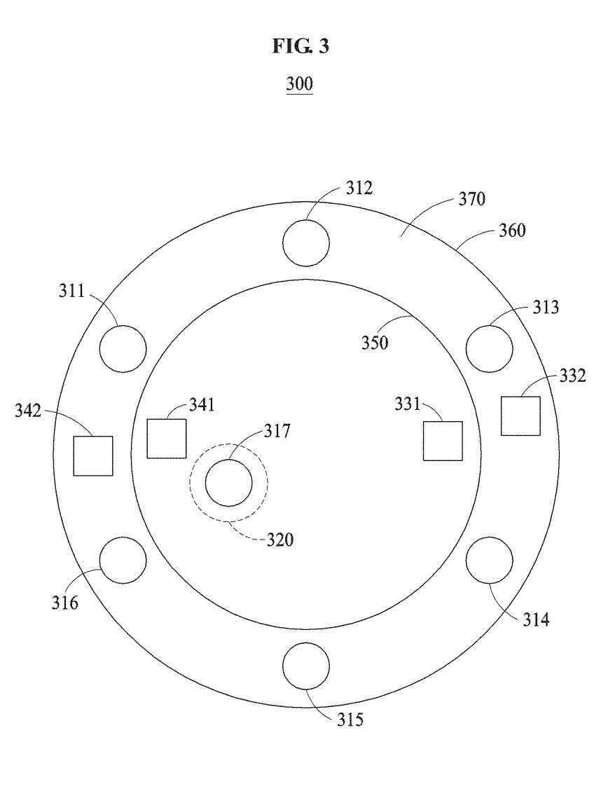

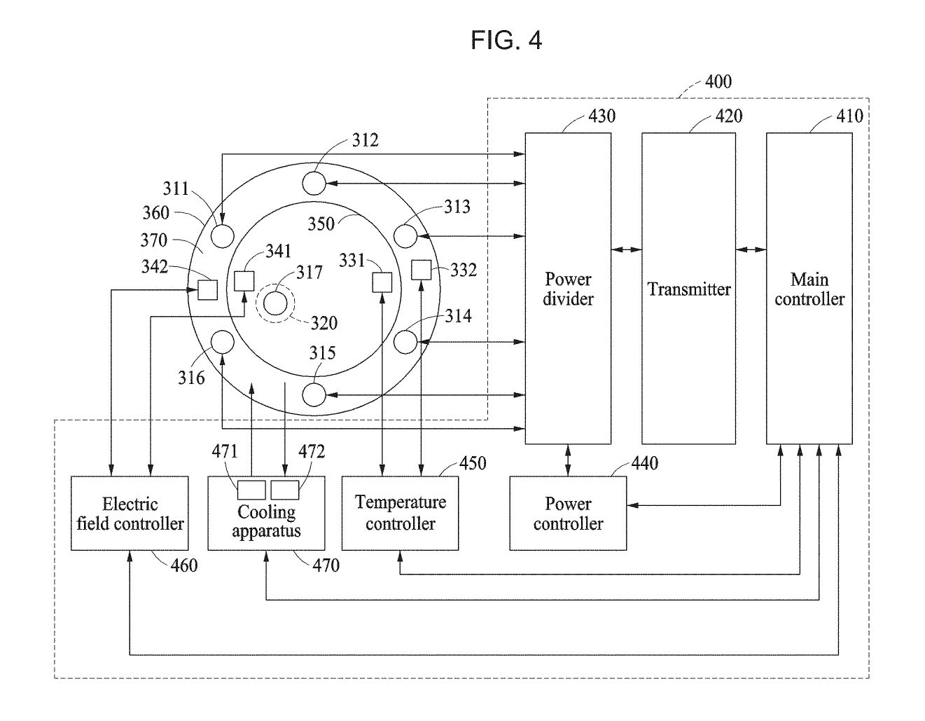

Hereinafter, description about an operation of the signal obtaining apparatus 200 is provided with reference to FIGS. 2 and 3, and description about an operation of the focusing apparatus 400 is provided with reference to FIG. 4.

FIG. 2 is a block diagram illustrating the signal obtaining apparatus 200 of FIG. 1 according to an example embodiment, and FIG. 3 illustrates the antenna array 300 of FIG. 1 according to an example embodiment.

In FIG. 3, it is assumed that a number of antennas included in the antenna array 300 is seven for ease of description. That is, the antenna array 300 includes a plurality of antennas 311 through 317.

Referring to FIGS. 2 and 3, the signal obtaining apparatus 200 includes an analysis module 210 and a mapping module 230.

The analysis module 210 may receive a signal obtained through an antenna of the antenna array 300 and a mapping model obtained from the mapping module 230, and may generate forward analysis data by performing a forward analysis.

When the analysis module 210 performs the forward analysis, the antenna 317 within a target point 320 may perform a role of a transmission antenna, and the antennas 311 through 316 outside the target point 320 may perform roles of reception antennas.

For example, the analysis module 210 obtains a signal passing through (or penetrating) the mapping model through the antennas 311 through 316 by transmitting a microwave to the mapping model corresponding to the target point 320 of an object 350 through the antenna 317. The analysis module 210 may generate forward analysis data based on a time reversal (TR) algorithm using the obtained signal. The forward analysis data may include data associated with an amplitude and/or a phase of the obtained signal.

The analysis module 210 may output the forward analysis data to the focusing apparatus 400. The analysis module 210 may be provided in a simulation-based analysis solver.

The mapping module 230 receives input data, for example, human body model data, associated with the object 350 from the focusing apparatus 400. The mapping module 230 may select a portion of the object 350 including the target point 320, and generate the mapping model by mapping the input data to the selected portion. For example, the mapping module 230 may fetch the input data from a data storing apparatus (not shown) of the focusing apparatus 400 and map the input data to a two-dimensional (2D) phantom model through a 2D model complier solver.

The mapping module 230 may output the mapping model to the analysis module 210.

The antenna array 300 includes the transmission and reception antennas 311 through 316, the target point 320, temperature sensors 331 and 332, electric field sensors 341 and 342, the object 350, a structure 360, and a background medium 370.

When the focusing apparatus 400 to be described below performs a reverse analysis, the antenna 317 positioned at the target point 320 may be removed and the antennas 311 through 316 positioned outside the target point 320 may perform roles of transmission antennas. That is, the antennas 311 through 317 may perform roles of transmission antennas and reception antennas through the forward analysis and the reverse analysis.

When the forward analysis is performed, signal data may be obtained by performing simulation using a mapping model and an analysis solver. When the reverse analysis is performed, a signal may be output to a target point using an actual hardware device. When the forward analysis is performed, a hardware device may be unnecessary.

Thus, the transmission antenna and the reception antenna are required when the forward analysis is performed, but the transmission antenna of the target point used for the forward analysis is removed when the reverse analysis is performed. When the reverse analysis is performed, a role of a plurality of outside reception antennas used for performing the forward analysis may be changed to be a role of a plurality of transmission antennas such that a signal is output toward a target point.

The antennas 311 through 317 may be provided in a form of an array including a dipole antenna, a monopole antenna in a patch form, and a waveguide antenna based on a shape of the structure 360 and a mechanical structure and a shape of the object 350. The antennas 311 through 317 may be provided in a form of a single layer or a multiple layer. The antenna array 300 may be contained in the background medium 370 or positioned outside the background medium 370, but this is not limited thereto.

Although FIG. 3 illustrates that the temperature sensor 331 and the electric field sensor 341 are positioned inside the object 350 and the temperature sensor 332 and the electric field sensor 341 are positioned outside the object 350, the temperature sensors 331 and 332, and the electric field sensors 341 and 342 are not limited thereto. The temperature sensors 331 and 332 and the electric field sensors 341 and 342 may be disposed in a place where a measurement is desired. Also, each sensor may be provided in a form of a probe.

An antenna array and an object may be inserted into the structure 360, and the background medium 370 may be put into the structure 360. A form of the structure 360 may be changed to be other forms based on a mechanical structure and a measurement mechanism. For example, the structure 360 may be provided in a form of a water tank and a form of a cylinder.

The background medium 370 may be matching liquid or cooling medium for cooling a boundary surface of the object 350 and the structure 360.

FIG. 4 is a block diagram illustrating the focusing apparatus 400 of FIG. 1 according to an example embodiment.

Referring to FIG. 4, the focusing apparatus 400 includes a main controller 410, a transmitter 420, a power divider 430, a power controller 440, a temperature controller 450, an electric field controller 460, and a cooling apparatus 470.

The main controller 410 may control an overall operation of the focusing apparatus 400. The main controller 410 may control an operation of each of constituent elements, for example, the transmitter 420, the power divider 430, the power controller 440, the temperature controller 450, the electric field controller 460, and the cooling apparatus 470, of the focusing apparatus 400. For example, the main controller 410 may be a personal computer (PC) and include a data storing apparatus. The PC may integrally control a system in a graphical user interface (GUI) program.

The main controller 410 may perform a reverse analysis based on forward analysis data received by the signal obtaining apparatus 200, and output a control signal for controlling the antenna array 300 based on a result of the reverse analysis.

The main controller 410 may monitor an object while outputting a microwave. For example, the main controller 410 may monitor a heat and a temperature using the temperature controller 450, and monitor an electric field intensity using the electric field controller 460.

The transmitter 420 may receive an output command of a signal from the main controller 410 and distribute signal data to be allocated to each of the antennas 311 through 316 to the power divider 430 and the power controller 440. The signal data may include an amplitude and a phase of the signal to be transmitted through each of the antennas 311 through 316.

The power divider 430 may distribute the power to a plurality of antennas included in the antenna array 300 by receiving the signal from the power controller 440 and transmitter 420.

The power controller 440 may control the power divider 430 by receiving the signal from the main controller 410.

The temperature controller 450 may control the temperature sensors 331 and 332, receive a result of measuring a temperature from the temperature sensors 331 and 332 to monitor a change in the heat and the temperature of an object, and transmit the result of the measuring to the main controller 410.

The electric field controller 460 may control the electric field sensors 341 and 342, receive a result of measuring an intensity of an electric field from the electric field sensors 341 and 342 to monitor the intensity of electric field to be focused on an object, and transmit the result of the measuring to the main controller 410.

The cooling apparatus 470 includes a background medium supplying apparatus 471 and a background medium discharging apparatus 472. The cooling apparatus 470 may cool an object through circulation of the background medium 370 in response to a control of the main controller 410. For example, the cooling apparatus 470 includes a connecting hose (not shown) for supplying and discharging the background medium 370, a pumping apparatus (not shown), an additional water tank (not shown) disposed outside to store the background medium 370, and an apparatus (not shown) for circulating the background medium 370 to cool a heat generated in an area excluding a target point due to an output of a microwave.

The cooling apparatus 470 may cool a heat generated on a boundary surface, for example, skin, of the background medium 370 and the object 350.

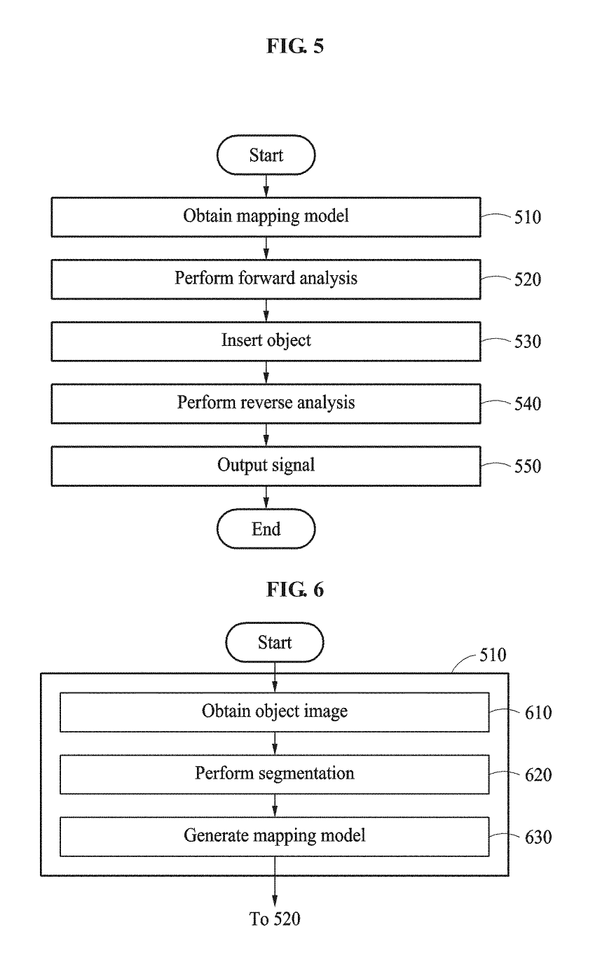

FIG. 5 is a flowchart illustrating a microwave output method according to an example embodiment.

Referring to FIG. 5, in operation 510, the mapping module 230 obtains a mapping model for an object. The mapping module 230 may receive an image required for mapping from the main controller 410, generate the mapping model by performing mapping on a cross-section of the object, and transmit the mapping model to the analysis module 210. For example, the mapping model is a phantom model.

In operation 520, the analysis module 210 performs a forward analysis for obtaining signal data from the antenna array 300 by receiving the mapping model from the mapping module 230. Here, the analysis module 210 may store a result of the forward analysis and transmit the result to the focusing apparatus 400. For example, a plurality of antennas are provided, and the signal data includes an amplitude and a phase of a signal.

In operation 530, the object 150 is inserted into the structure 360 of the antenna array 300. An insertion of the object 150 may be manually performed by a person, or automatically performed by an additional device or a machine. The insertion may be performed by various methods based on shapes of the object 150 and the structure 360.

In operation 540, the main controller 410 performs a reverse analysis based on the mapping model received by the signal obtaining apparatus 200 and the result of the forward analysis result. The main controller 410 may perform the reverse analysis based on a time reversal (TR) algorithm. A condition for the reverse analysis may be set in the main controller 410, and the condition may include an operational frequency, and an operation, a communication, a transmission power, and an output time of each control apparatus. For example, the operational frequency may correspond to 925 megahertz (MHz) or 2.45 gigahertz (GHz) which is one of industry-science-medical (ISM) bands.

When the reverse analysis is performed, an amplitude value may be determined based on an attenuation of an amplitude of a signal to be output in response to the background medium 370 and the object 150 being loss mediums having conductivity, and a frequency loss occurring when a signal, for example, a microwave, to be output returns to the target point 320. For example, the determined amplitude value may be double an input amplitude value.

In addition, the main controller 410 can use a threshold value for an amplitude value of a determined signal. The threshold is to prevent tissue damage of the surrounding tissue when beam focused on the target point 320. The threshold value can be set to an initial threshold value of 1 since the amplitude value of the determined signal is different for each receiving antenna but has a maximum value of 1 or less. Also, an output phase may be determined based on the signal data obtained in an operation of performing the forward analysis. For example, the phase is determined by applying an opposite sign to the signal phase obtained in the operation of performing the forward analysis.

Based on a result of the reverse analysis, an amplitude and a phase of a signal and a power allocated by the main controller 410 through the transmitter 420 and the power divider 430 may have different values for each of the transmission antennas 311 through 316 based on a position of each of the transmission antennas 311 through 316.

The main controller 410 may store the result of the reverse analysis and output a control signal to each of the transmitter 420, the power divider 430, the power controller 440, the temperature controller 450, the electric field controller 460, and the cooling apparatus 470.

In operation 550, the antennas 311 through 316 performing transmission roles of the antenna array 300 output a signal, for example, a microwave, to the target point 320 based on the control signal allocated by the focusing apparatus 400. The main controller 410 may output the control signal to the power divider 430 through the transmitter 420 based on the result of the reverse analysis, and the power divider 430 may divide the power to each of the transmission antennas 311 through 316. Each of the transmission antennas 311 through 316 may output a microwave to the target point 320 using the divided power. Each of the transmission antennas 311 through 316 may output the microwave to the target point 320 at the same time or at different points in time.

Each of the transmission antennas 311 through 316 may allow the microwave to be focused on the target point 320 thereby performing thermotherapy. The target point 320 may indicate an affected area, for example, a tumor, of an object, and the tumor may be necrotized through the thermotherapy.

FIG. 6 is a flowchart illustrating a mapping model obtaining method according to an example embodiment.

Referring to FIG. 6, in operation 610, the mapping module 230 obtains an object image from the main controller 410. The mapping module 230 may perform mapping by inputting the obtained image to a two-dimensional (2D) complier solver. The 2D complier solver may map the input image to a 2D phantom model. In addition, the object image may be a voxel phantom model including a magnetic resonance imaging (MRI) based image file or a computed tomography (CT) based image file. The voxel phantom model may be associated with a whole human body or a predetermined portion of the human body.

In operation 620, the mapping module 230 performs segmentation to clarify the target point 320 from the obtained image. In an operation of performing the segmentation, the mapping module 230 may generate a 2D image by selecting a cross-section of the obtained image and set a mapping condition. The mapping condition includes a position of a target point inside the phantom model, a conductivity and a permittivity of a medium outside the phantom model, and a mesh cell size of the phantom model for a spatial resolution.

In operation 630, the mapping module 230 generates the mapping model based on a difference between a conductivity and a permittivity of each portion of an object by receiving the mapping condition and the 2D image obtained through the segmentation, and represents an area corresponding to the target point. The mapping module 230 may store the generated mapping model and transmit data associated with the generated mapping model to the analysis module 210.

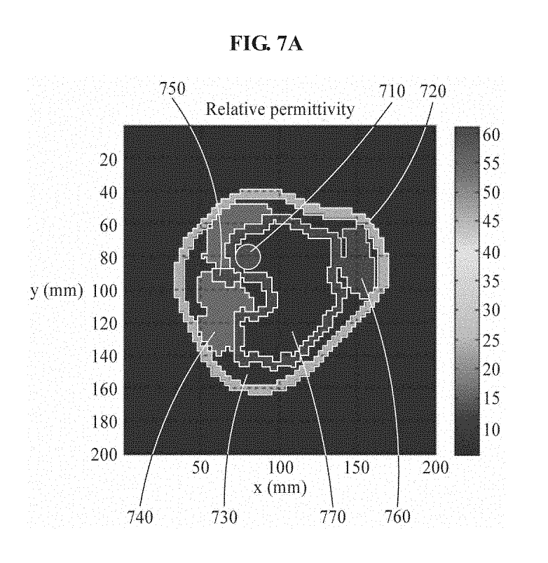

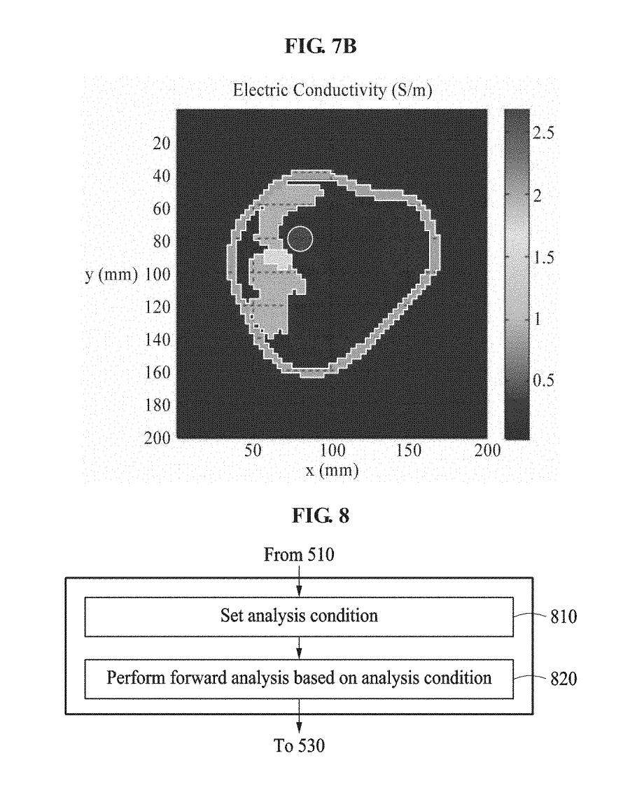

FIG. 7A illustrates a permittivity mapping model according to an example embodiment, and FIG. 7B illustrates a conductivity mapping model according to an example embodiment. Referring to FIG. 7A, a position of a tumor 710 corresponding to the target point 320 is at an 80-millimeter point on an x-axis and an 80-millimeter point on a y-axis. The tumor 710 indicates an osteosarcoma in bone marrow and is ten millimeters in diameter. An outermost boundary portion indicates a skin texture 720, a dark portion neighboring the skin texture 720 indicates a fat tissue 730, a left portion indicates a muscle tissue 740, and a portion between left portions indicating the muscle tissue 740 indicates a blood vessel 750, a portion around the tumor 710 indicates a bone (integument) 760, and an inner portion of the bone 760 indicates a bone marrow 770. An outer portion of the skin texture 720 indicates the background medium 370.

The conductivity mapping model of FIG. 7B represents tissues identical to those of the permittivity mapping model of FIG. 7A. Right bars in FIGS. 7A and 7B indicate values of a permittivity and a conductivity.

FIG. 8 is a flowchart illustrating a forward analysis according to an example embodiment.

Referring to FIG. 8, in operation 810, the analysis module 210 sets an analysis condition based on a mapping model received from the mapping module 230. The analysis module 210 may fetch the mapping model transmitted from the mapping module 210 to a forward analysis solver by executing the forward analysis solver, and set a forward analysis condition, for example, a setting of the antenna 317 performing a role of a transmission antenna of the target point 320, settings of the antennas 311 through 316 performing roles of reception antennas outside the mapping model, a setting of a signal frequency, a setting of an analysis time, and a setting of a signal type, based on the fetched mapping model. For example, the analysis module 210 may dispose the antenna 317 at the target point 320, and dispose the antennas 311 through 316 outside the target point 320. The analysis module 210 may set a 925 megahertz (MHz) frequency, one transmission antenna, 16 reception antennas, 10 nanoseconds of analysis time, and a continuous wave (CW) signal.

In operation 820, the analysis module 210 performs a forward analysis based on the set analysis condition. The analysis module 210 may be provided by a simulation-based solver and perform a simulation based on the received mapping model. The antenna 317 in a simulation may transmit a signal to each of the antennas 311 through 316, and each of the antennas 311 through 316 may obtain signal data from a signal passing through the object 350 and the background medium 370. For example, the signal data may include an amplitude and a phase of the signal. The analysis module 210 may store the obtained signal data and transmit the signal data to the focusing apparatus 400.

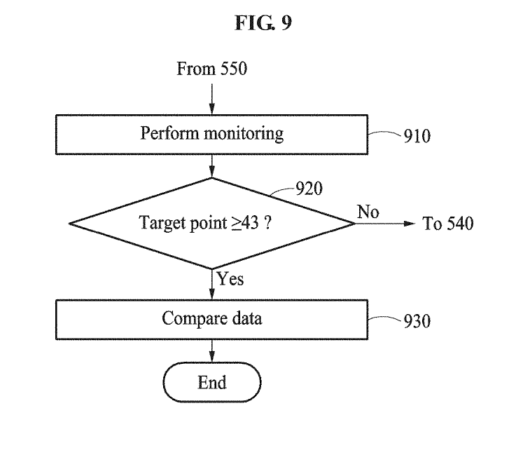

FIG. 9 is a flowchart illustrating a method using a microwave output result according to an example embodiment.

Referring to FIG. 9, in operation 910, the main controller 410 monitors an inside and an outside of an object while outputting a microwave. For example, the main controller 410 measures a change in a heat and a temperature and an electric field intensity of the inside and the outside of the object using the electric field sensors 341 and 342 and the temperature sensors 331 and 332. The main controller 410 may perform cooling through the background medium 370 in response to a temperature of an area excluding the target point 332 being greater than or equal to a first reference value. For example, the first reference value corresponds to 42□. Each of the power controller 440 and the temperature controller 450 may transmit a monitoring result to the main controller 410, and the main controller 410 may store the monitoring result.

In operation 920, the main controller 410 verifies whether the temperature of the target point 320 is greater than or equal to a reference value based on the monitoring result. The main controller 410 may terminate an output of a signal in response to the temperature of the target point 320 increasing by a second reference value while the transmission antennas 311 through 316 are outputting the signal, may reset a measurement time and a measurement condition in response to the temperature of the target point 320 not increasing by the second reference value, and may set an output condition by re-performing the reverse analysis (operation 540). For an example, the second reference value corresponds to 43□.

Also, the main controller 410 may stop outputting when a normal tissue is damaged in response to the temperature of the area excluding the target point 320 being greater than or equal to the first reference value while outputting the signal, may fix a phase value of a next signal after resetting a reverse analysis condition, and may output the microwave by readjusting threshold value for amplitude values only. When the amplitude values are readjusted, a damage of the tissue occurring in response to the temperature of the area excluding the target point 320 being greater than or equal to the first reference value may be prevented. The main controller 410 may store monitoring data when an output of the signal terminates.

In operation 930, the main controller 410 compares the monitoring data to simulation data when the output of signal terminates. The main controller 410 may terminate a thermotherapy process by comparing the stored monitoring data to the simulation data generated by performing simulation.

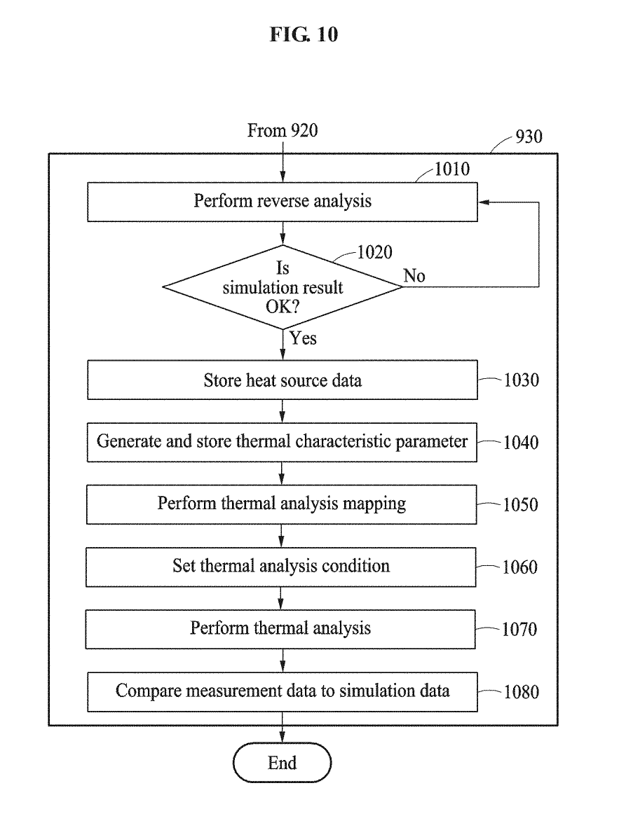

FIG. 10 is a flowchart illustrating a method of comparing a measurement result to a simulation result according to an example embodiment.

Referring to FIG. 10, in operation 1010, the main controller 410 performs a reverse analysis simulation based on stored signal data and a mapping model. The main controller 410 executes a reverse analysis solver and fetches the mapping model to perform a reverse analysis. The main controller 410 sets an analysis condition, for example, an analysis frequency, an analysis time, a number of transmission antennas, and a signal type, based on a method identical to that of a forward analysis. For example, an analysis frequency is set to be 925 MHz, a number of transmission antennas is set to be 16, an analysis time is set to be 10 nanoseconds, and a signal type is set to be a continuous wave (CW) signal. When a condition is set, the main controller 410 may perform a simulation that simultaneously outputs signals to the target point 320 based on an amplitude value and a phase value of a signal allocated to each of the transmission antennas 311 through 316 using the fetched mapping model. Here, the main controller 410 may store a result of the simulation that outputs the signals.

In operation 1020, the main controller 410 receives and analyzes the stored result of the simulation. For example, a simulation result includes a power absorption density (PAD), a specific absorption rate (SAR), and an E-field distribution characteristic that recognize whether a microwave is output and focused on a target point. Here, when a result of analyzing the SAR and the PAD indicates a relatively great value in an area excluding the target point 320 inside the mapping model, the main controller 410 decreases a characteristic value of the area excluding the target point 320 and re-performs the reverse analysis in order to have a great value only in the target point 320. In this case, an amplitude value of a signal allocated to each of the transmission antennas 311 through 316 is decreased to be less than or equal to a threshold value and a signal output simulation is performed without changing a phase.

In operation 1030, the main controller 410 stores a heat source data from the result of reverse analysis to use the heat source data for a thermal analysis when the reverse analysis simulation is complete. The heat source data includes PAD data.

In operation 1040, the main controller 410 generates and stores a thermal characteristic parameter associated with each portion of an object to perform the thermal analysis. Table 1 represents an example of the thermal characteristic parameter for the thermal analysis.

TABLE 1

| | | | | Bloodflow, | Metabolic |
|---|---|---|---|---|---|
| Whole | | Thermal | Heat | Heat | Rate, Heat |
| body | Density | Conductivity | Capacity | Transfer Rate | Generation Rate |
| models | [kg/m³] | [W/m/□] | [J/kg/□] | [W/K/m³] | [W/m³] |
| Name | Tissue | Internal heat | Tissue Specific | Capillary Blood | Metabolic Heat |
| | Density | Conduction | Heat Capacity | Perfusion | Conduction |
| Blood | 1,049.75 | 0.5169 | 3,617.00 | 709,090 | 0 |
| Bone | | | | | |
| Cortical | 1,908.00 | 0.3200 | 1,312.83 | 1,288.83 | 154.87 |
| Bone | | | | | |
| Marrow | 980 | 0.1921 | 2,065 | 1,985.93 | 464.61 |
| Fat | 911.00 | 0.2115 | 2,348.33 | 2,012.82 | 506.57 |
| Muscle | 1,090.40 | 0.4950 | 3,421.20 | 2,705.95 | 906.12 |
| Skin | 1,109.00 | 0.3722 | 3,500.00 | 7,969.16 | 1,647.52 |
| Tumor | 1,040.00 | 0.560 | 3,437 | 51,000 | 12,000 |

A thermal characteristic parameter may include a degree of autonomously generating a heat (metabolic heat conduction), a characteristic of losing a heat (blood flow and blood perfusion), a heat capacity, a thermal conductivity, and a density of an object. The thermal characteristic parameter may be used for the thermal analysis.

In operation 1050, the main controller 410 performs thermal analysis mapping based on permittivity and conductivity mapping data obtained by the electromagnetic analysis, and the generated heat source data. The generated thermal analysis mapping model is stored in a storing apparatus of the main controller 410. The main controller 410 may convert an electromagnet mapping model used to output a microwave into a thermal analysis mapping model, and generate a mapping model for the thermal analysis by assigning an identification (ID) to each tissue of the mapping model. The generated mapping model may correspond to the thermal characteristic parameter through the assigned ID, and fetch thermal parameter information for the thermal analysis.

In operation 1060, the main controller 410 sets a thermal analysis condition when the thermal analysis mapping is complete. The thermal analysis condition includes an exposure time, an initial temperature of a thermal analysis mapping model, and a temperature and a power of the background medium 370. For example, an exposure time is set to be five minutes, a temperature of the background medium 370 is set to be 30□, and an initial temperature of the thermal analysis mapping model is set to be 36□, and a power is set to be 10 watts (W).

In operation 1070, the main controller 410 performs the thermal analysis based on the set condition by executing a thermal analysis solver. When the main controller 410 increases a power corresponding to the target point of the thermal analysis mapping model in the simulation, an inside temperature of the thermal analysis mapping model may increase from the initial temperature. As a result, a convection current may occur between the background medium 370 and the thermal analysis mapping model. An analysis terminates when the temperature of the target point increases above a temperature of a neighboring portion and then increases by a target temperature. An initial temperature of the mapping model may be 36□, an initial temperature of the background medium 370 may be 30□, and a target temperature may be 43□. The main controller 410 may output and store the result when the thermal analysis terminates.

In operation 1080, the main controller 410 compares measurement data obtained based on a result of outputting the microwave to data obtained through simulation, and the thermal analysis terminates.

The components described in the exemplary embodiments of the present invention may be achieved by hardware components including at least one DSP (Digital Signal Processor), a processor, a controller, an ASIC (Application Specific Integrated Circuit), a programmable logic element such as an FPGA (Field Programmable Gate Array), other electronic devices, and combinations thereof. At least some of the functions or the processes described in the exemplary embodiments of the present invention may be achieved by software, and the software may be recorded on a recording medium. The components, the functions, and the processes described in the exemplary embodiments of the present invention may be achieved by a combination of hardware and software.

The processing device described herein may be implemented using hardware components, software components, and/or a combination thereof. For example, the processing device and the component described herein may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A microwave output method comprising:
generating a mapping model corresponding to an object by mapping at least one of a permittivity or a conductivity to an image of the object; and
performing a forward analysis on a target point based on a time reversal (TR) algorithm using the mapping model for allowing a signal to be focused on the target point of the object through a plurality of antennas.

2. The method of claim 1, wherein the performing of the forward analysis comprises:
setting a condition for a transmission antenna and a reception antenna based on the mapping model; and
measuring a signal received by the reception antenna based on the condition.

3. The method of claim 2, wherein the setting comprises disposing the transmission antenna at the target point.

4. The method of claim 2, wherein the setting comprises disposing the reception antenna outside the target point, and the reception antenna is provided as a plurality of antennas.

5. The method of claim 2, wherein the condition includes at least one of a position of the transmission antenna, a position of the reception antenna, a frequency of the signal, a type of the signal, or an analysis time.

6. The method of claim 1, wherein the performing of the forward analysis comprises representing an area corresponding to the target point to the mapping model based on the permittivity or the conductivity.

7. A microwave output method comprising:
receiving signals received from a target point of an object; and
outputting a signal to the target point from each of a plurality of antennas by performing a reverse analysis on the received signals based on a time reversal (TR) algorithm.

8. The method of claim 7, wherein the outputting comprises determining at least one of an amplitude or a phase of the signal based on at least one of an attenuation or a loss.

9. The method of claim 8, wherein the determining comprises determining the amplitude of the signal to be less than or equal to a threshold value set to prevent surrounding tissue damage of the target point and determining the phase of the signal as opposed to a phase of the receiving signals received from the target point of the object.

10. The method of claim 9, further comprising:
monitoring a temperature of the object.

11. The method of claim 10, further comprising:
decreasing the temperature in response to a temperature of an area excluding the target point in the object being greater than or equal to a first reference value by readjusting the threshold value; and
re-performing the reverse analysis based on the TR algorithm by changing a condition in response to a temperature of the target point in the object being less than or equal to a second reference value.

12. A microwave output method comprising:
performing a forward analysis based on a time reversal (TR) algorithm using a mapping model corresponding to a target point of an object;
performing a reverse analysis based on the TR algorithm based on forward analysis data for the forward analysis; and
outputting a signal to the target point from each of a plurality of antennas based on reverse analysis data for the reverse analysis.

13. The method of claim 12, wherein the performing of the forward analysis comprises:
setting a condition for a transmission antenna and a reception antenna based on the mapping model; and
measuring a signal received by the reception antenna based on the condition.

14. The method of claim 13, wherein the setting comprises disposing the transmission antenna at the target point.

15. The method of claim 13, wherein the setting comprises disposing the reception antenna outside the target point, and the reception antenna is provided as a plurality of antennas.

16. The method of claim 13, wherein the condition includes at least one of a position of the transmission antenna, a position of the reception antenna, a frequency of the signal, a type of the signal, or an analysis time.

17. The method of claim 12, wherein the performing of the forward analysis comprises:
generating the mapping model by mapping a permittivity or a conductivity to an image of the object; and
representing an area corresponding to the target point to the mapping model based on the permittivity or the conductivity.

18. The method of claim 12, wherein the performing of the reverse analysis comprises determining at least one of an amplitude or a phase of the signal based on at least one of an attenuation or a loss.

19. The method of claim 18, wherein the determining comprises determining the amplitude of the signal to be less than or equal to a threshold value set to prevent surrounding tissue damage of the target point and determining the phase of the signal as opposed to a phase of the receiving signals received from the target point of the object.

20. The method of claim 19, further comprising:
decreasing the temperature in response to a temperature of an area excluding the target point in the object being greater than or equal to a first reference value by readjusting the threshold value; and
re-performing the reverse analysis based on the TR algorithm by changing a condition in response to a temperature of the target point in the object being less than or equal to a second reference value.
